(19) United States
(12) Patent Application Publication
(10) Pub. No.: US 2016/0235299 A1
(43) Pub. Date: Aug. 18, 2016

(54) OPHTHALMIC SURGICAL MICROSCOPE AND OPHTHALMIC SURGICAL ATTACHMENT

(71) Applicant: Kabushiki Kaisha Topcon, Itabashi-ku (JP)

(72) Inventors: Satoshi YAMAMOTO, Saitama-shi (JP); Takefumi HAYASHI, Wako-shi (JP)

(73) Assignee: Kabushiki Kaisha Topcon, Itabashi-ku (JP)

(21) Appl. No.: 14/995,313

(22) Filed: Jan. 14, 2016

(30) Foreign Application Priority Data
Feb. 16, 2015 (JP) 2015-027489

(51) Int. Cl. A61B 3/18; A61B 3/10; A61B 3/135

(52) U.S. Cl. CPC A61B 3/185; A61B 3/135; A61B 3/102

(57) ABSTRACT

An ophthalmic surgical microscope of an embodiment includes an objective lens, illumination system, observation system, OCT system, and optical-path connecting member. The illumination system includes a diaphragm irradiated with light from an illumination light source and a lens unit including one or more lenses that make the light having passed through the diaphragm into a parallel light flux, and irradiates the light having passed through the lens unit to an eye through the objective lens. The observation system is configured for observing the eye being irradiated by the illumination system through the objective lens. The OCT system is configured for examining the eye by OCT through the objective lens. The optical-path connecting member is located between the diaphragm and the lens unit or between the lens unit and the objective lens to connect the optical path of the OCT system to that of the illumination system.

### CROSS-REFERENCE TO RELATED APPLICATION

This application is based upon and claims the benefit of priority from Japanese Patent Application No. 2015-027489, filed 16 Feb. 2015; the entire contents of which are incorporated herein by reference.

### FIELD

Embodiments described herein relate generally to an ophthalmic surgical microscope and an ophthalmic surgical attachment.

### BACKGROUND

In the ophthalmic field, various types of surgeries are performed. Among them are cataract surgery and vitreoretinal surgery, for example. An ophthalmic surgical microscope is used in such surgery. The ophthalmic surgical microscope is used for visual observation of an eye through an observation system illuminated by an illumination system or capturing of images.

There are such ophthalmic surgical microscopes that are provided with an optical coherence tomography (OCT) device (see, for example, Japanese Unexamined Patent Application Publication Nos. 2008-264488, 2008-264490, 2008-268852, 2009-230141, and 2008-264489). The OCT device is used to capture cross sectional images and three-dimensional images, measure the size of tissue (the thickness of a layer, etc.), acquire functional information (blood flow information, etc.), and the like.

The OCT device is required to be capable of obtaining an OCT signal of sufficient intensity as well as having a high resolution, a wide scan range, and a compact structure. To satisfy these requirements, an important factor is the position for coupling the OCT optical path with the optical path of the ophthalmic surgical microscope.

In the surgical microscope disclosed in Japanese Unexamined Patent Application Publication Nos. 2008-264488, 2008-264490, 2008-268852, and 2009-230141, the OCT optical path is connected to the observation optical path. In the surgical microscope disclosed in Japanese Unexamined Patent Application Publication No. 2008-264489, the OCT optical path is connected to the illumination optical path, and these optical paths are connected to the observation optical path.

As disclosed in Japanese Unexamined Patent Application Publication Nos. 2008-264488, 2008-264490, 2008-268852, and 2009-230141, if the OCT optical path is connected to the observation optical path, sufficient resolution cannot be achieved due to a restriction in the diameters of the lenses arranged in the observation optical path. The OCT optical path may be connected to the observation optical path at a position between the objective lens and the eye to avoid this problem. In this case, however, the device cannot be compact and may interfere with the manipulation or operation of the surgeon. Besides, in the configuration disclosed in Japanese Unexamined Patent Application Publication No. 2008-264489, the OCT optical path is connected to the illumination optical path at a position between lenses that constitute a lens unit for making illumination light into a parallel light flux. This results in a complicated optical design. Thus, it becomes difficult to downsize the surgical microscope and modularize the OCT device attachable to the surgical microscope.

### SUMMARY

Embodiments are intended to solve the above problems, and the object is to provide an ophthalmic surgical microscope and an ophthalmic surgical attachment capable of wide-range and high-resolution OCT examination with a compact structure.

According to one embodiment, an ophthalmic surgical microscope includes an objective lens, illumination system, observation system, OCT system, and optical-path connecting member. The illumination system includes a diaphragm irradiated with light from an illumination light source and a lens unit including one or more lenses that make the light having passed through the diaphragm into a parallel light flux, and irradiates the light having passed through the lens unit to an eye through the objective lens. The observation system is configured for observing the eye being irradiated by the illumination system through the objective lens. The OCT system is configured for examining the eye by OCT through the objective lens. The optical-path connecting member is located between the diaphragm and the lens unit or between the lens unit and the objective lens to connect the optical path of the OCT system to that of the illumination system.

### BRIEF DESCRIPTION OF THE DRAWINGS

FIG. 1 is a schematic diagram illustrating an example of the configuration of an optical system of an ophthalmic surgical microscope according to a first embodiment.

FIG. 2 is a schematic diagram illustrating an example of the configuration of the optical system of the ophthalmic surgical microscope of the first embodiment.

FIG. 3 is a schematic diagram illustrating an example of the configuration of an optical system of an ophthalmic surgical microscope according to a second embodiment.

FIG. 4A is a diagram illustrating an example of the operation of the ophthalmic surgical microscope of the second embodiment.

FIG. 4B is a diagram illustrating an example of the operation of the ophthalmic surgical microscope of the second embodiment.

FIG. 5A is a diagram illustrating an example of the operation of the ophthalmic surgical microscope of the second embodiment.

FIG. 5B is a diagram illustrating an example of the operation of the ophthalmic surgical microscope of the second embodiment.

FIG. 6 is a schematic diagram illustrating an example of the configuration of an optical system of an ophthalmic surgical microscope according to a third embodiment.

FIG. 7 is a schematic diagram illustrating an example of the configuration of an optical system of an ophthalmic surgical microscope according to a fourth embodiment.

FIG. 8A is a diagram illustrating an example of the operation of the ophthalmic surgical microscope of the fourth embodiment.

FIG. 8B is a diagram illustrating an example of the operation of the ophthalmic surgical microscope of the fourth embodiment.

FIG. 9 is a schematic diagram illustrating an example of the configuration of an optical system of an ophthalmic surgical microscope according to a fifth embodiment.

FIG. 10 is a schematic diagram illustrating an example of the configuration of an optical system of an ophthalmic surgical microscope according to a sixth embodiment.

FIG. 11 is a schematic diagram illustrating an example of the configuration of an optical system of an ophthalmic surgical microscope according to a seventh embodiment.

FIG. 12A is a diagram illustrating an example of the operation of the ophthalmic surgical microscope of the seventh embodiment.

FIG. 12B is a diagram illustrating an example of the operation of the ophthalmic surgical microscope of the seventh embodiment.

### DETAILED DESCRIPTION

Referring now to the drawings, a description is given of examples of embodiments of an ophthalmic surgical microscope and an ophthalmic surgical attachment. The ophthalmic surgical microscope of the following embodiments is used in ophthalmic surgery. The ophthalmic surgical microscope is a device that illuminates an eye (a patient's eye) by an illumination system so that the light returning therefrom enters the observation system to capture an observation image of the eye. The ophthalmic surgical attachment is configured to be detachably attached to the ophthalmic surgical microscope.

In the following embodiments, the ophthalmic surgical microscope becomes capable of OCT examination when equipped with an ophthalmic surgical attachment that includes at least part of the OCT optical system. It is assumed herein that the OCT examination includes the acquisition of cross sectional images and/or three-dimensional images, the measurement of the size of tissue (the thickness of a layer, etc.), the acquisition of functional information (blood flow information, etc.), and the like. The target site to be examined by OCT may be any site of the eye. Examples of the site include the cornea, vitreous body, crystalline lens, and ciliary body in the anterior segment of the eye, and the retina, choroid, and vitreous body in the posterior segment. The target site may also be a periphery of the eye such as the eyelid and eye socket. By a known method, it is possible to form a cross sectional image or a three-dimensional image of the eye based on measurement light returning through the ophthalmic surgical attachment.

Hereinafter, the measurement light for OCT examination and the light returning from the eye may be collectively referred to as OCT light. In addition, images acquired by OCT may be collectively referred to as OCT images. Further, measurement for forming an OCT image may be referred to as OCT measurement. Incidentally, the contents of documents cited above may be incorporated by reference herein.

In the following embodiments, a configuration using a Fourier domain OCT is described. In particular, the ophthalmic surgical microscope (and ophthalmic surgical attachment) of the following embodiments is capable of the OCT examination of the eye using a known swept-source OCT technology.

The embodiments may be applied also to ophthalmic surgical microscopes other than those using a swept-source OCT, such as, for example, those using a spectral domain OCT. Although the following embodiments describe a device combining an OCT device that includes an optical system of OCT and an ophthalmic surgical microscope, the OCT device of the embodiments may be combined with an ophthalmologic observation device other than the ophthalmic surgical microscope, such as, for example, a scanning laser ophthalmoscope (SLO), a slit lamp, and a fundus camera.

### First Embodiment

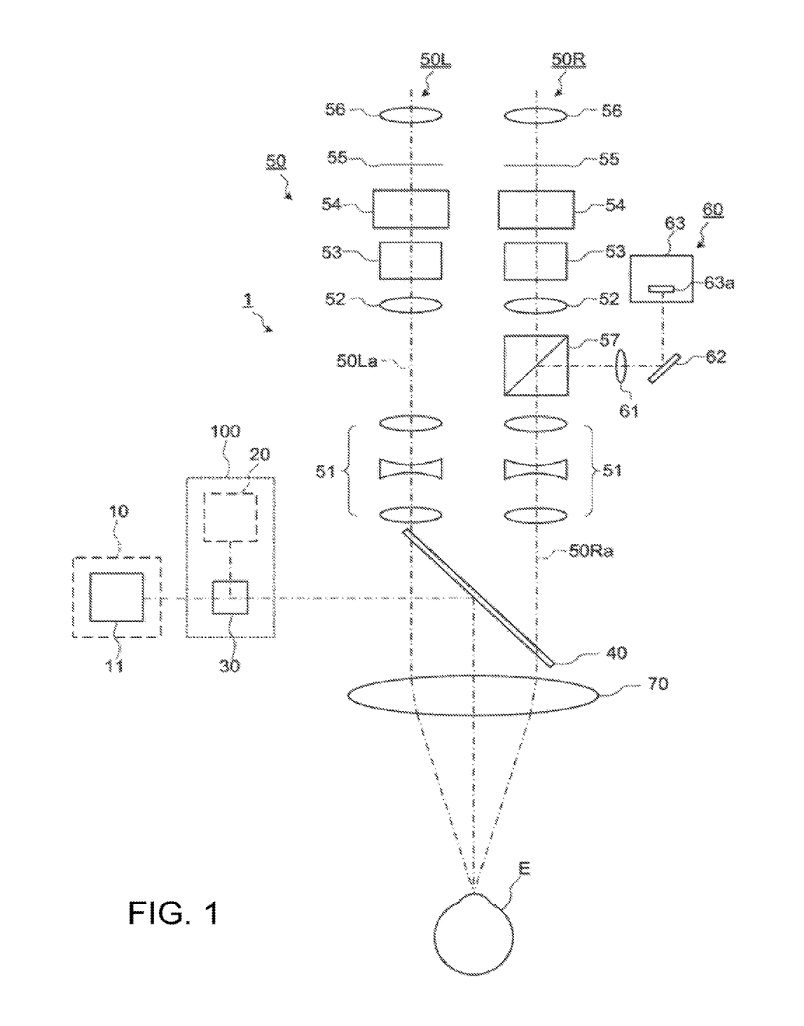

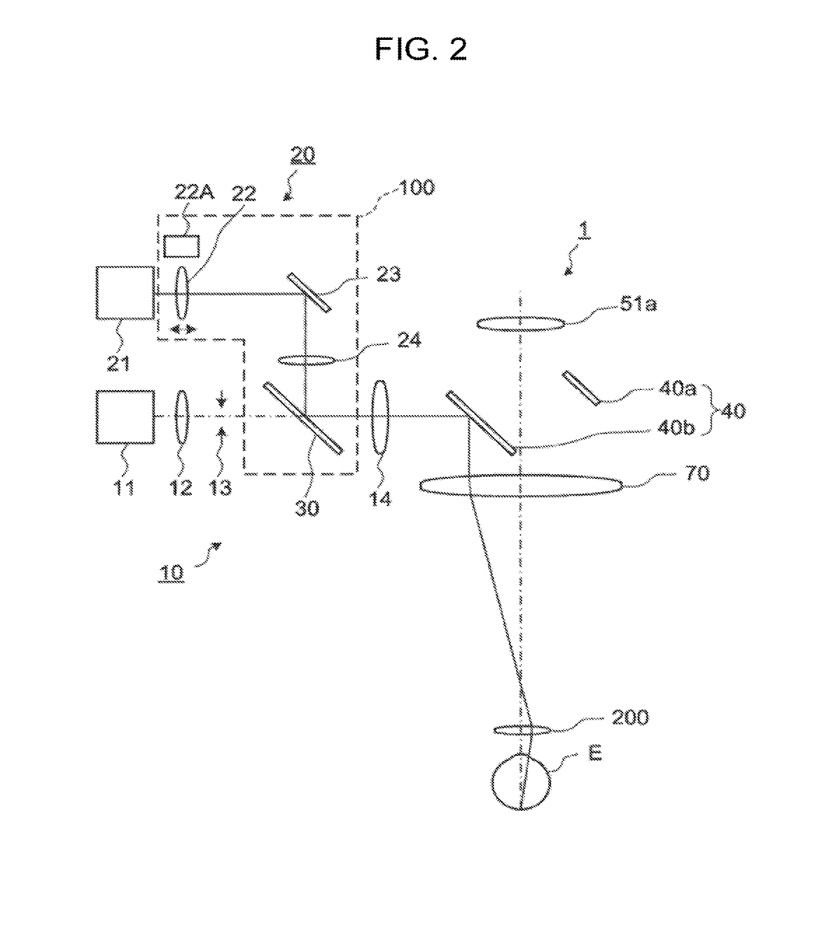

FIGS. 1 and 2 illustrate the optical system of an ophthalmic surgical microscope 1 according to the first embodiment. FIG. 1 illustrates the configuration of the optical system of an observation system viewed from the operator side. FIG. 2 illustrates a side view of the optical system of an illumination system and an interference optical system of FIG. 1 viewed from the operator. Like reference numerals designate like parts in FIGS. 1 and 2. Incidentally, in addition to the configuration illustrated in FIGS. 1 and 2, the ophthalmic surgical microscope may be provided with an optical system (assistant microscope) for the operator's assistant to observe an eye E.

In the present embodiment, directions such as upper and lower, left and right, front and back, and the like are defined as viewed from the operator side unless otherwise noted. Regarding the upper and lower directions, the direction from the objective lens toward the observation object (eye E) is referred to as “lower”, and the opposite direction is referred to as “upper”. In general, patient undergoes surgery in the supine position, and thus the upper and lower directions correspond to the vertical direction.

The optical system of the ophthalmic surgical microscope 1 includes an illumination system 10, an OCT system 20, an optical-path connecting member 30, a deflector 40, an observation system 50, and an objective lens 70. The ophthalmic surgical microscope 1 further includes an ophthalmic surgical attachment 100 that is configured to be detachably attached to the ophthalmic surgical microscope 1. At least part of the OCT system 20 (e.g., collimating lens 22, optical scanner 23, OCT lens 24) and the optical-path connecting member 30 are provided in the ophthalmic surgical attachment 100. In the following, the illumination system 10, the OCT system 20, the optical-path connecting member 30, and the deflector 40 are described mainly with reference to FIG. 2. The observation system 50 is described mainly with reference to FIG. 1.

The ophthalmic surgical microscope 1 may include a front lens 200 that is configured to be removably inserted into a position on the optical axis of the objective lens 70 as a main objective lens. The front lens 200 can be placed in a position between the front focal position of the objective lens 70 and the eye E. The front lens 200 focuses the light from the illumination system 10 to illuminate the inside of the eye E (the posterior eye segment such as the retina, vitreous body, etc.). The front lens 200 includes a plurality of lenses having different refractive powers (e.g., 40D, 80D, 120D, etc.), which are selectively used. FIG. 1 illustrates the front lens 200 retracted from the position on the optical axis of the objective lens 70. FIG. 2 illustrates the front lens 200 inserted into the position on the optical axis of the objective lens 70.

### (Illumination System)

The illumination system 10 includes an illumination light source 11, a condenser lens 12, and a diaphragm 13 (illumination field diaphragm, visual field diaphragm), and a lens unit 14. The illumination light source 11 emits illumination light including visible light. The condenser lens 12 condenses the illumination light emitted from the illumination light source 11. The diaphragm 13 limits the illumination field of the illumination light condensed by the condenser lens 12. The diaphragm 13 is located in a position optically conjugate with the front focal position of the objective lens 70. The lens unit 14 includes one or more lenses that make the light having passed through the diaphragm 13 into a parallel light flux. While FIG. 2 illustrates an example in which the lens unit 14 includes one lens, the lens unit 14 may include two or more lenses. The illumination light having passed through the lens unit 14 is irradiated onto the eye E through the objective lens 70 of the observation system 50.

### (OCT System)

The OCT system 20 includes an optical system for examining the eye E by using OCT through the objective lens 70. The OCT system may have the same configuration as the conventional Fourier domain OCT device (e.g., swept-source OCT). The OCT system 20 includes an interference optical system 21, a collimating lens 22, a focus adjustment mechanism 22A, an optical scanner 23, and an OCT lens 24.

The light from the OCT light source is split into measurement light and reference light. The interference optical system 21 emits the measurement light, and causes the measurement light returning from the eye to interfere with the reference light to generate interference light. The interference optical system 21 includes, for example, a splitter part, and an interference part. The splitter part splits the light from the OCT light source (e.g., wavelength-swept light source (wavelength tunable light source)) into measurement light and reference light. The OCT light source emits light at near-infrared wavelengths invisible to the human eye. The measurement light is emitted toward the eye E. The reference light is emitted toward a predetermined reference optical path. The interference part causes the measurement light returning from the eye E to interfere with the reference light having passed through the reference optical path, thereby generating interference light. The interference light generated by the interference optical system 21 is detected by a detector (not illustrated). The detector obtains detection signals and sends them to an arithmetic and control unit (not illustrated). Like conventional swept-source OCT devices, the arithmetic and control unit applies arithmetic processing such as Fourier transform to the detection signals. Note that the interference optical system 21 may include an OCT light source.

For example, one end of an optical fiber is connected to the interference optical system 21. The other end of the optical fiber is located in a position facing the collimating lens 22. The measurement light emitted from the interference optical system 21 is guided by the optical fiber to be incident on the collimating lens 22. The return light of the measurement light that has passed through the collimating lens 22 is guided by the optical fiber to be incident on the splitter part of the interference optical system 21.

The collimating lens 22 collimates the measurement light emitted from the interference optical system 21 into a parallel light flux. The focus adjustment mechanism 22A moves the collimating lens 22 along the optical axis of the OCT system 20. The focus adjustment mechanism 22A includes, for example, a holding member, a slide mechanism, and an actuator. The holding member holds the collimating lens 22. The slide mechanism is configured to move the holding member in the direction along the optical axis of the OCT system 20. The actuator generates a driving force. The focus adjustment mechanism 22A further includes a member configured to transmit the driving force to the slide mechanism. The focus adjustment mechanism 22A can move the collimating lens 22 manually or automatically. In the case of manual operation, the focus adjustment mechanism 22A controls the actuator based on operation on an operation unit (not illustrated) performed by a user (e.g., operator) to thereby move the collimating lens 22. In the case of automatic operation, a control unit (not illustrated) controls the actuator so that the measurement light returning from the eye E, the interference light, and/or the detection signal has an intensity above a predetermined value, and thus the focus adjustment mechanism 22A can move the collimating lens 22.

The optical scanner 23 two-dimensionally deflects the measurement light, which has been collimated into a parallel light flux by the collimating lens 22. Thereby, the optical scanner 23 scans the eye E with the measurement light collimated into a parallel light flux. The optical scanner 23 includes, for example, a first galvanometer mirror and a second galvanometer mirror. The first galvanometer mirror deflects the measurement light in a first direction in the scan plane set for the eye E. The second galvanometer mirror deflects the measurement light in a second direction perpendicular to the first direction. The optical scanner 23 further includes a mechanism for driving them independently. In this case, the optical scanner 23 can scan the eye E with the measurement light in arbitrary directions on a plane defined by the first and second directions.

The OCT lens 24 is arranged between the optical scanner 23 and the optical-path connecting member 30, and functions as a variable power lens. The measurement light deflected by the optical scanner 23 passes through the OCT lens 24, and is guided to the optical-path connecting member 30.

The optical-path connecting member 30 is arranged between the diaphragm 13 and the lens unit 14 on the optical path of the illumination system 10, and connects the optical path of the OCT system 20 to the optical path of the illumination system 10. Specifically, the optical-path connecting member 30 is arranged to face a lens of the one or more lenses constituting the lens unit 14, which is optically closest to the illumination light source 11. In the present embodiment, the optical axis of the OCT system 20 and that of the illumination system 10 are arranged coaxially. For example, the OCT system 20 and the illumination system 10 are arranged such that the positions of their optical axes match each other in the reflective surface of the optical-path connecting member 30. As an example of the optical-path connecting member 30 may be cited a dichroic mirror. The collimating lens 22, the focus adjustment mechanism 22A, the optical scanner 23, the OCT lens 24, and the optical-path connecting member 30 are provided in the ophthalmic surgical attachment 100. Further, at least part of the interference optical system 21 may be provided in the ophthalmic surgical attachment 100. Furthermore, the OCT light source may be provided in the ophthalmic surgical attachment 100.

The deflector 40 is arranged between the optical-path connecting member 30 and the objective lens 70, and deflects the light in the optical paths of the illumination system 10 and the OCT system 20 toward the objective lens 70. The deflector 40 may be included in the observation system 50. Examples of the deflector 40 include a beam splitter, a half mirror, a dichroic mirror, and an epi-illumination mirror formed of one or more reflecting members. FIG. 1 illustrates the deflector 40 as a beam splitter. FIG. 2 illustrates the deflector 40 as an epi-illumination mirror formed of two reflecting members 40a and 40b.

As a beam splitter, the deflector 40 is arranged in the optical path of the observation system 50. In this case, the beam splitter (the deflector 40) coaxially connects the optical path of the observation system 50 and the optical path of the OCT system 20.

As an epi-illumination mirror, the deflector 40 is desirably arranged outside the optical path of the observation system 50. In FIG. 2, the light having passed through the lens unit 14 is reflected by the reflecting members 40a and 40b. The reflecting member 40a is arranged to reflect the light having passed through the lens unit 14 and not reflected by the reflecting member 40b.

### (Observation System)

The observation system 50 includes an optical system for observing the eye E, through the objective lens 70, being illuminated by the illumination system 10. As illustrated in FIG. 1, the observation system 50 includes a pair of left and right observation systems 50L and 50R, and an imaging optical system 60. Hereinafter, the observation system 50L on the left side is referred to as “left observation system” (left observation optical axis 50La), while the observation system 50R on the right side is referred to as “right observation system” (right observation optical axis 50Ra). The left and right observation systems 50L and 50R are arranged to sandwich the optical axis of the objective lens 70.

The left and right observation systems 50L and 50R each include a variable power lens system 51, an imaging lens 52, an erecting prism 53, a pupil distance adjustment prism 54, a visual field diaphragm 55, and an eyepiece 56. The right observation system 50R is provided with a beam splitter 57 between the variable power lens system 51 and the imaging lens 52.

The variable power lens system 51 includes a plurality of zoom lenses 51a, 51b, and 51c. Each of the zoom lenses 51a to 51c is movable by a zooming mechanism (not illustrated) in a direction along the left observation optical axis 50La or the right observation optical axis 50Ra. Thereby, the magnification for observing or photographing the eye E is changed.

The beam splitter 57 separates part of observation light guided along the right observation optical axis 50Ra from the eye E and leads it to the imaging optical system 60. The imaging optical system 60 includes an imaging lens 61, a reflecting mirror 62, and an imaging unit 63.

The imaging unit 63 includes an imaging device 63a. The imaging device 63a may be formed of, for example, a charge coupled device (CCD) image sensor, a complementary metal oxide semiconductor (CMOS) image sensor, or the like. For the imaging device 63a, those having a two-dimensional light receiving surface (area sensor) are used.

In the use of the ophthalmic surgical microscope 1, for example, the light receiving surface of the imaging device 63a is located in a position optically conjugate with the surface of the cornea of the eye E, or a position optically conjugate with a position separated from the corneal apex in the depth direction by a half of the radius of curvature of the cornea.

The erecting prism 53 turns an image right-side up. The pupil distance adjustment prism 54 is an optical element for adjusting the distance between left observation light and right observation light according to the operator's eye width (distance between the left and right eyes). The visual field diaphragm 55 shields a peripheral region in the cross section of the observation light to limit the field of view of the operator.

In the above configuration, regardless of whether the ophthalmic surgical attachment 100 is being attached, the ophthalmic surgical microscope 1 irradiates the eye E with illumination light to enable the observation of the eye E. Specifically, the illumination light output from the illumination light source 11 is condensed by the condenser lens 12. The illumination light then passes through the diaphragm 13 and the optical-path connecting member 30, and is collimated into a parallel light flux by the lens unit 14. The illumination light collimated into a parallel light flux is deflected by the deflector 40. After passing through the objective lens 70, the illumination light is irradiated to the eye E. When the front lens 200 is inserted to a position on the optical axis of the objective lens 70, the illumination light deflected by the deflector 40 passes through the objective lens 70 and the front lens 200, and then is irradiated to the eye E.

The illumination light (part of the illumination light) irradiated to the eye E is reflected by the cornea or within the eye. The illumination light reflected by the eye E (sometimes referred to as “observation light”) travels through the objective lens 70 (in some cases, the front lens 200) and is incident on the observation system 50. With this configuration, a magnified image of the eye E can be observed through the eyepiece 56. An image captured by the imaging unit 63 may be displayed on a display (not illustrated).

When the ophthalmic surgical attachment 100 is attached to the ophthalmic surgical microscope 1, the optical-path connecting member 30 is located between the diaphragm 13 and the lens unit 14. The measurement light obtained by dividing the light from the OCT light source travels through the collimating lens 22, the optical scanner 23, and the OCT lens 24, and is reflected by the optical-path connecting member 30. The measurement light reflected by the optical-path connecting member 30 travels through the lens unit 14, and after being deflected by the deflector 40, passes through the objective lens 70 (in some cases, also the front lens 200), thereby reaching the eye E. The measurement light reflected by the eye E returns therefrom through the same path as described above to the interference optical system 21. The interference optical system 21 causes the measurement light returning from the eye to interfere with the reference light obtained by splitting the light from the OCT light source to generate interference light. The interference light generated by the interference optical system 21 is detected by a detector (not illustrated). The detector obtains detection signals and sends them to the arithmetic and control unit (not illustrated). Like conventional swept-source OCT devices, the arithmetic and control unit applies arithmetic processing such as Fourier transform to the detection signals. Based on the result of the arithmetic processing, the arithmetic and control unit forms a cross sectional image or a three-dimensional image of a predetermined site of the eye E, measures the size of tissue (the thickness of a layer, etc.), generates functional information (blood flow information, etc.), and the like.

In the present embodiment, the aperture (diameter) of the optical element, through which the measurement light of the OCT system 20 and the return light of the measurement light (OCT light) pass, can be increased. Accordingly, it is possible to enlarge the scan range by the measurement light and the numerical aperture affecting the resolution of the measurement light of the OCT system 20. Besides, the optical path of the OCT system 20 is connected to the optical path of the illumination system 10 at a position between the diaphragm 13 and the lens unit 14. Thereby, the illumination system 10 and the OCT system 20 can share the lens unit 14. This results in a reduced number of optical elements, thus realizing a compact device.

In general, a relatively large physical space can be secured between the diaphragm 13 and the lens unit 14. Therefore, by modularizing the collimating lens 22, the optical scanner 23, the OCT lens 24, and the optical-path connecting member 30, it is possible to achieve an ophthalmic surgical attachment (100) that can be easily attached to and detached from the ophthalmic surgical microscope 1 without influence on the optical system such as the positional deviation and the axial displacement of the diaphragm 13 due to vibration, or the like. Incidentally, there may be provided an adjustment mechanism for correcting the positional deviation and the axial displacement of the diaphragm 13, or the like.

In the present embodiment, an example is described in which at least part of the OCT system 20 and the optical-path connecting member 30 are modularized as the ophthalmic surgical attachment 100; however, they are not necessarily be modularized.

### Operations and Effects

Described below are the operations and effects of the ophthalmic surgical microscope and the ophthalmic surgical attachment according to the first embodiment.

According to the first embodiment, an ophthalmic surgical microscope (e.g., the ophthalmic surgical microscope 1) includes an objective lens (e.g., the objective lens 70), an illumination system (e.g., the illumination system 10), an observation system (e.g., the observation system 50), an OCT system (e.g., the OCT system 20), and an optical-path connecting member (e.g., the optical-path connecting member 30). The illumination system includes a diaphragm (e.g., the diaphragm 13) and a lens unit (e.g., the lens unit 14), and irradiates light having passed through the lens unit to an eye (e.g., the eye E) through the objective lens. The diaphragm is irradiated with light from a light source. The lens unit includes one or more lenses configured to collimate the light having passed through the diaphragm into a parallel light flux. The observation system is used to observe the eye being illuminated by the illumination system through the objective lens. The OCT system is used to examine the eye through the objective lens by using OCT. The optical-path connecting member is located between the diaphragm and the lens unit, and connects the optical path of the OCT system to the optical path of the illumination system.

With this configuration, in the ophthalmic surgical microscope used for the surgery of the eye, the aperture (diameter) of the optical element, through which the measurement light of the OCT system passes, can be increased. Accordingly, it is possible to widely design the scan range with the measurement light and the numerical aperture affecting the resolution of the measurement light of the OCT system. Besides, the optical path of the OCT system is connected to the optical path of the illumination system at a position between the diaphragm and the lens unit. Thereby, the illumination system and the OCT system can share the lens unit. This results in a reduced number of optical elements of the illumination system, thus realizing a compact device. Further, since the optical path of the OCT system is connected to the optical path of the illumination system, wide-range OCT examination can be performed with a high resolution.

The ophthalmic surgical microscope may further include a deflector (e.g., the deflector 40), which is located between the optical-path connecting member and the objective lens, and deflects light in the optical path of the illumination system and light in the optical path of the OCT system toward the objective lens.

With this configuration, the eye can be illuminated from the objective lens side, thus achieving a compact device.

Further, the deflector may include a beam splitter arranged on the optical path of the observation system.

With this configuration, the deflector for deflecting the illumination light and the light of the OCT system can be placed in the observation optical path, thus achieving a compact device.

The beam splitter may coaxially connect the optical path of the observation system and the optical path of the OCT system with each other.

With this configuration, OCT examination can be performed on the eye in a condition close to a state where the eye is observed by the observation system.

In addition, the OCT system may include an interference optical system (e.g., the interference optical system 21), a collimating lens (e.g., the collimating lens 22), an optical scanner (e.g., the optical scanner 23), and an OCT lens (e.g., the OCT lens 24). The light from the OCT light source is split into measurement light and reference light. The interference optical system emits the measurement light, and causes the measurement light returning from the eye to interfere with the reference light to generate interference light. The collimating lens collimates the measurement light emitted from the interference optical system into a parallel light flux. The optical scanner two-dimensionally deflects the measurement light, which has been collimated into a parallel light flux by the collimating lens. The OCT lens is located between the optical scanner and the optical-path connecting member.

With this configuration, OCT examination can be performed with the ophthalmic surgical microscope used for the surgery of the eye by employing a known method.

The ophthalmic surgical microscope may further include a focus adjustment mechanism (e.g., the focus adjustment mechanism 22A) configured to move the collimating lens along the optical axis of the OCT system.

With this configuration, in the ophthalmic surgical microscope used for the surgery of the eye, high-resolution OCT examination can be performed in the optimum conditions.

The ophthalmic surgical microscope may further include an ophthalmic surgical attachment (e.g., the ophthalmic surgical attachment 100) configured to be detachably attached to the ophthalmic surgical microscope. The collimating lens, the optical scanner, the OCT lens, and the optical-path connecting member may be provided in the ophthalmic surgical attachment.

In this configuration, since the collimating lens, the optical scanner, the OCT lens, and the optical-path connecting member are modularized to be detachably attached to the ophthalmic surgical microscope, the ophthalmic surgical microscope can be switched to a device capable of OCT examination.

According to the first embodiment, an ophthalmic surgical attachment is configured to be detachably attached to an ophthalmic surgical microscope for examining the eye through the objective lens by OCT. The ophthalmic surgical attachment includes a collimating lens, an optical scanner, an OCT lens, and an optical-path connecting member. When the ophthalmic surgical attachment is attached to the ophthalmic surgical microscope, the optical-path connecting member may be located between the diaphragm and the lens unit. The ophthalmic surgical microscope includes an objective lens and an illumination system. The illumination system includes a diaphragm irradiated with light from a light source, and a lens unit having one or more lenses configured to collimate the light having passed through the diaphragm into a parallel light flux, and is configured to irradiate the light having passed through the lens unit to the eye through the objective lens. The ophthalmic surgical microscope includes an observation system used to observe, through the objective lens, the eye being illuminated by the illumination system. The interference optical system splits the light from the OCT light source into measurement light and reference light, emits the measurement light, and causes the measurement light returning from the eye to interfere with the reference light to generate interference light. The collimating lens collimates the measurement light emitted from the interference optical system into a parallel light flux. The optical scanner two-dimensionally deflects the measurement light, which has been collimated into a parallel light flux by the collimating lens. The measurement light deflected by the optical scanner passes through the OCT lens. The optical-path connecting member is used to connect the optical path of the measurement light having passed through the OCT lens to the optical path of the illumination system.

With this configuration, the collimating lens, the optical scanner, the OCT lens, and the optical-path connecting member can be modularized as an ophthalmic surgical attachment that is easily attached to and detached from the ophthalmic surgical microscope. In general, a relatively large physical space can be secured between the diaphragm and the lens unit as well as between the lens unit and the objective lens. Thus, it is possible to switch the ophthalmic surgical microscope to a device capable of OCT examination without influence on the optical system such as the positional deviation and the axial displacement of the diaphragm, or the like.

### Second Embodiment

In the first embodiment, the illumination optical path and the OCT optical path are connected coaxially. Therefore, the deflector 40 and the iris (pupil) of the eye E may cause vignetting in the measurement light from the OCT system 20 and the like. In particular, if the deflector 40 is formed of an epi-illumination mirror, since a reflecting member that constitutes the epi-illumination mirror has a complicated shape, vignetting may occur in the measurement light from the OCT system 20. When vignetting occurs in the measurement light, the range of OCT scan is limited, and the OCT system 20 cannot exert its full performance.

For this reason, in the second embodiment, the optical axis of the illumination system and the optical axis of the OCT system are arranged to be non-coaxial. The optical axis of the illumination system and the optical axis of the OCT system may be fixed to be non-coaxial, or they may be moved relatively to be adjusted as in the present embodiment.

An ophthalmic surgical microscope of the second embodiment has basically the same configuration as that of the first embodiment. In the following, the second embodiment is described with a focus on differences from the first embodiment.

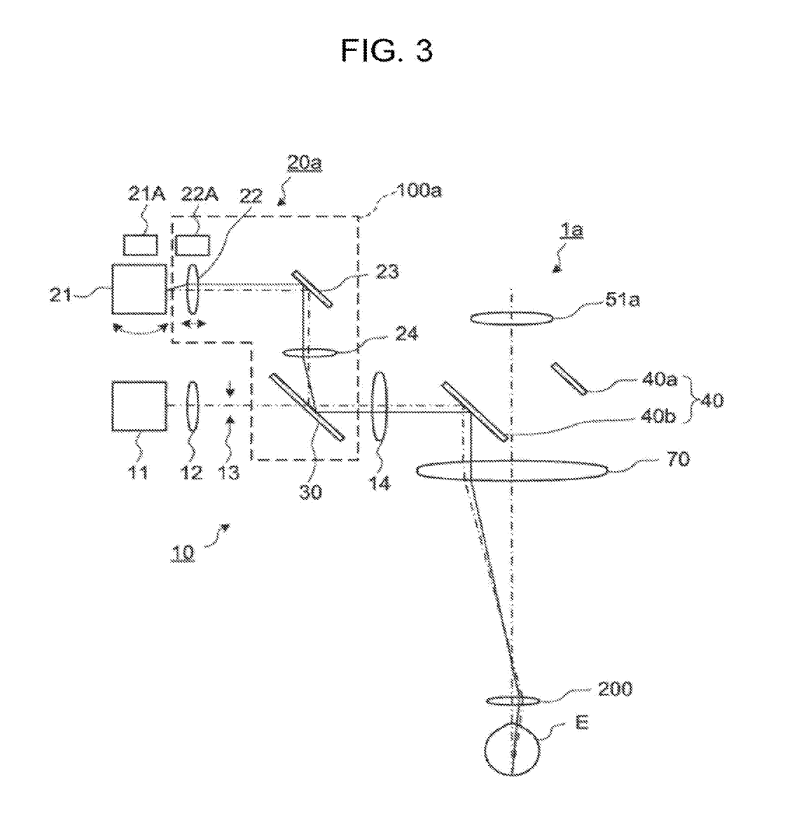

FIG. 3 illustrates the configuration of an optical system of an ophthalmic surgical microscope 1a of the second embodiment. In FIG. 3, like reference numerals designate like parts as in FIG. 2, and the redundant explanation may be omitted as appropriate.

The ophthalmic surgical microscope 1a of the second embodiment is different in configuration from the ophthalmic surgical microscope 1 of the first embodiment in the presence of an optical-axis adjustment mechanism 21A. An OCT system 20a includes the interference optical system 21, the optical-axis adjustment mechanism 21A, the collimating lens 22, the focus adjustment mechanism 22A, the optical scanner 23, and the OCT lens 24. An ophthalmic surgical attachment 100a includes the collimating lens 22, the focus adjustment mechanism 22A, the optical scanner 23, the OCT lens 24, and the optical-path connecting member 30. The ophthalmic surgical attachment 100a may further include the interference optical system 21 and the optical-axis adjustment mechanism 21A. The ophthalmic surgical attachment 100a may further include an OCT light source.

The optical-axis adjustment mechanism 21A moves the optical axis of the illumination system 10 and that of the OCT system 20 relative to each other. For example, the optical-axis adjustment mechanism 21A changes the direction of measurement light emitted from the interference optical system 21 to relatively move the optical axis of the illumination system 10 and that of the OCT system 20. The direction of measurement light emitted from the interference optical system 21 may be changed by tilting the end of an optical fiber for emitting the measurement light from the interference optical system 21 or tilting the case that holds an optical element constituting the interference optical system 21. When the end of the optical fiber is tilted, the optical-axis adjustment mechanism 21A includes a holding member, an emission angle deflector, an actuator that generates a driving force, and a member that transmits the driving force to the emission angle deflector. The holding member is configured to movably hold one end of an optical fiber having the other end connected to the interference optical system 21. The emission angle deflector is configured to move the holding member to change the emission angle of measurement light in reference to a predetermined emission direction. The optical-axis adjustment mechanism 21A is capable of moving the optical axis of the OCT system 20 toward the center of the pupil of the eye E by the above mechanism.

The optical-axis adjustment mechanism 21A moves the optical axis of the illumination system 10 (e.g., the center of a illumination light flux) and the optical axis of the OCT system 20 (e.g., the center of a measurement light flux) relative to each other such that they are located in different positions in the reflective surface of the deflector 40 (in the example of FIG. 3, the reflecting members 40a and 40b). As a result, the optical-path connecting member 30 connects the optical path of the OCT system 20 to the optical path of the illumination system 10 non-coaxially.

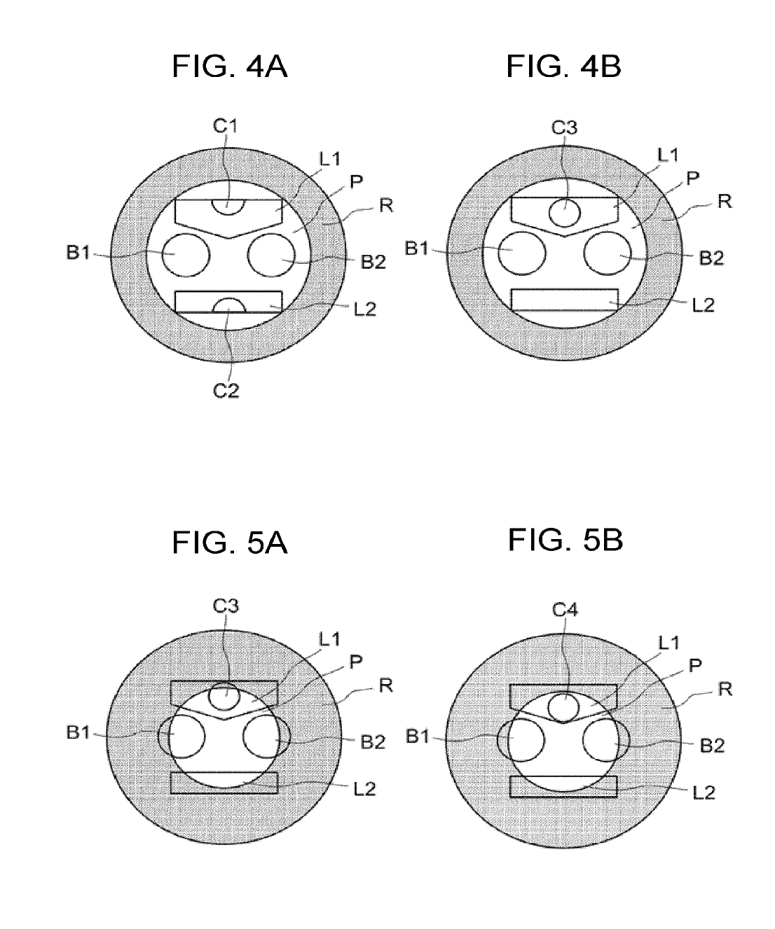

FIGS. 4A and 4B are diagrams for explaining the operation to observe the fundus of the eye E. To observe the fundus of the eye E, the front lens 200 is inserted in a position between the objective lens 70 and the eye E as illustrated in FIG. 3. FIG. 4A schematically illustrates the pupil of each optical system incident on the pupil of the eye E when vignetting occurs in measurement light due to the deflector 40 (epi-illumination mirror). FIG. 4B schematically illustrates the pupil of each optical system incident on the pupil of the eye E when the optical-axis adjustment mechanism 21A changes the direction of measurement light from the interference optical system 21. Like reference numerals designate like parts in FIGS. 4A and 4B, and the redundant explanation may be omitted as appropriate.

When the fundus of the eye E is observed, the illumination pupil (image) of the illumination system 10 and the observation pupil of the observation system 50 are placed in the pupil P of the eye E such that the iris R causes no vignetting. For example, an illumination pupil L1 of illumination light reflected by the reflecting member 40b in illumination light emitted from the illumination system 10, an illumination pupil L2 of illumination light reflected by the reflecting member 40a in illumination light emitted from the illumination system 10, an observation pupil B1 of the left observation system 50L, and an observation pupil B2 of the right observation system 50R are placed in the pupil P of the eye E as illustrated in FIG. 4A. Here, if the deflector 40 is formed of an epi-illumination mirror having a complicated shape, and an OCT pupil of the OCT system 20 is placed in the pupil P, for example, an OCT pupil C1 of the OCT system 20 may be arranged to overlap the illumination pupil L1, and an OCT pupil C2 of the OCT system 20 may be arranged to overlap the illumination pupil L2. In this case, vignetting occurs in the measurement light of the OCT system 20 due to the deflector 40 (epi-illumination mirror).

In the present embodiment, when the optical-axis adjustment mechanism 21A changes the direction of measurement light emitted from the interference optical system 21, as illustrated in FIG. 4B, an OCT pupil C3 of the OCT system 20 can be arranged to overlap the illumination pupil L1. Thereby, it is possible to suppress the occurrence of vignetting of measurement light due to the deflector 40. Thus, the OCT system 20 can exert its full performance without limiting the range of OCT scan.

FIGS. 5A and 5B are diagrams for explaining the operation to observe the fundus of the eye E having a small pupil. FIG. 5A schematically illustrates the pupil of each optical system incident on the pupil of the eye E when the iris of the eye E as a small pupil causes vignetting in measurement light. FIG. 5B schematically illustrates the pupil of each optical system incident on the pupil of the eye E when the optical-axis adjustment mechanism 21A changes the direction of measurement light from the interference optical system 21. In FIGS. 5A and 5B, like reference numerals designate like parts as in FIGS. 4A and 4B, and the redundant explanation may be omitted as appropriate.

When the eye E has a small pupil, the OCT pupil C3 of the OCT system 20 is arranged as illustrated in FIG. 5A, and the iris R may cause vignetting in measurement light. Here, if the optical-axis adjustment mechanism 21A changes the direction of measurement light emitted from the interference optical system 21, as illustrated in FIG. 5B, an OCT pupil C4 of the OCT system 20 can be arranged to overlap the illumination pupil L1 in the pupil P. Thereby, it is possible to suppress the occurrence of vignetting of measurement light due to the iris R. Thus, the OCT system 20 can exert its full performance without limiting the range of OCT scan. This is particularly effective when the pupil of the eye E is small at the time of observing the fundus, and the position adjustment of the device is not sufficient.

In the present embodiment, while the optical-axis adjustment mechanism 21A is described as adjusting the optical axis of the OCT system 20, the direction of illumination light emitted from the illumination system 10 may be changed to move the optical axis of the illumination system 10 and the optical axis of the OCT system 20 relative to each other.

### Operations and Effects

According to the second embodiment, an ophthalmic surgical microscope may include an optical-axis adjustment mechanism (e.g., the optical-axis adjustment mechanism 21A) configured to move the optical axis of the illumination system and that of the OCT system relative to each other.

With this configuration, it is possible to suppress the occurrence of vignetting in the light of the OCT system due to the deflector or the iris of the eye. Thus, the OCT system can exert its full performance without limiting the range of OCT scan.

According to the embodiment, the light from the OCT light source is split into measurement light and reference light. The interference optical system emits the measurement light, and causes the measurement light returning from the eye to interfere with the reference light to generate interference light. The optical-axis adjustment mechanism may change the direction of the measurement light emitted from the interference optical system to relatively move the optical axis of the illumination system and that of the OCT system.

With this, the ophthalmic surgical microscope of the embodiment having a relatively simple structure can be provided with an OCT system that can exert its full performance.

### Third Embodiment

In the first embodiment or the second embodiment, an example is described in which the optical-path connecting member 30 is arranged between the diaphragm 13 and the lens unit 14; however, the configuration of the ophthalmic surgical microscope of the embodiment is not limited to this.

In the third embodiment, the optical-path connecting member 30 is arranged between the lens unit 14 and the objective lens 70. The optical axis of the illumination system and that of the OCT-system are positioned coaxially.

An ophthalmic surgical microscope of the third embodiment has basically the same configuration as that of the first embodiment. In the following, the third embodiment is described with a focus on differences from the first embodiment.

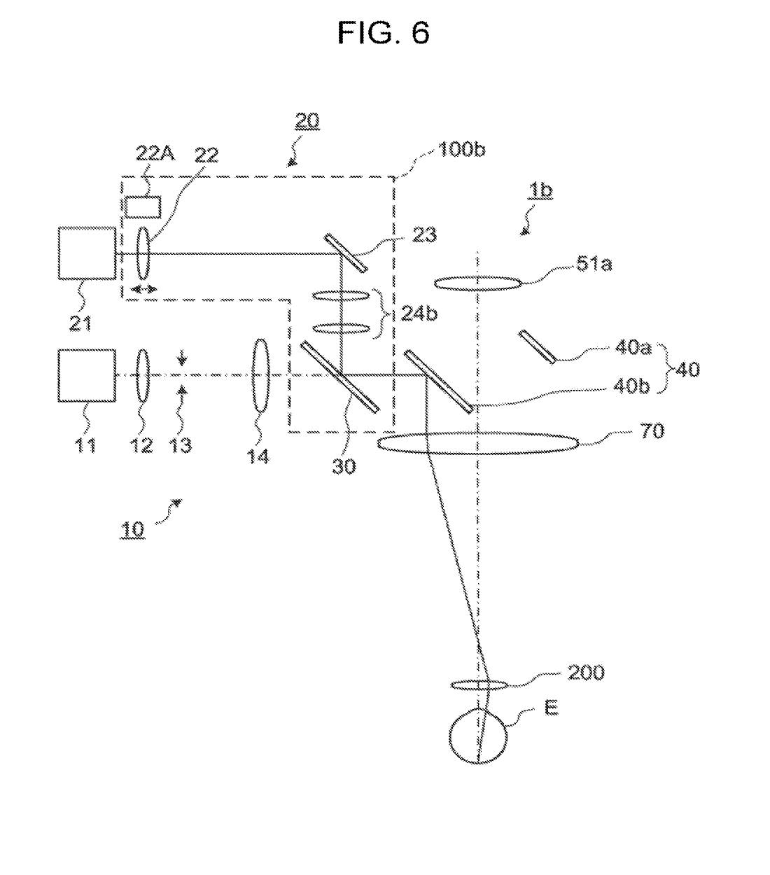

FIG. 6 illustrates the configuration of an optical system of an ophthalmic surgical microscope 1b of the third embodiment. In FIG. 6, like reference numerals designate like parts as in FIG. 2, and the redundant explanation may be omitted as appropriate.

The ophthalmic surgical microscope 1b of the third embodiment is mainly different in configuration from the ophthalmic surgical microscope 1 of the first embodiment in that the optical-path connecting member 30 is located between the lens unit 14 and the objective lens 70 (the deflector 40), and the presence of an OCT lens 24b formed of two or more lenses in place of the OCT lens 24.

The optical-path connecting member 30 is located between the lens unit 14 and the objective lens 70 on the optical path of the illumination system 10 to connect the optical path of the OCT system 20 to the optical path of the illumination system 10. Specifically, the optical-path connecting member 30 is arranged to face a lens of the one or more lenses constituting the lens unit 14, which is optically closest to the objective lens 70. Incidentally, in FIG. 6, the deflector 40 is arranged between the optical-path connecting member 30 and the objective lens 70.

In the third embodiment, the measurement light of the OCT system 20 is irradiated to the eye E without passing through the lens unit 14. Therefore, for example, by providing the OCT lens 24b as illustrated in FIG. 6, it is possible to achieve a design taking into account the transmission loss and the aberration of the measurement light. Thus, according to the present embodiment, OCT examination can be performed using measurement light from the OCT system 20 with improved accuracy as compared to the first embodiment in which the illumination system 10 and the OCT system 20 share the lens unit 14.

Further, according to the embodiment, the optical-path connecting member 30 is located on the parallel optical path where the illumination light of the illumination system 10 is made into a parallel light flux. Therefore, it is possible to reduce the influence on the illumination system 10 associated with the attachment/detachment of an ophthalmic surgical attachment 100b to/from the ophthalmic surgical microscope 1b (e.g., the positional displacement of the optical element of the illumination system 10, and the like).

### Operations and Effects

According to the third embodiment, an ophthalmic surgical microscope includes an objective lens, and an illumination system. The an illumination includes a diaphragm irradiated with light from a light source, and a lens unit having one or more lenses configured to collimate the light having passed through the diaphragm into a parallel light flux, and is configured to irradiate, through the objective lens, the light having passed through the lens unit to the eye. The ophthalmic surgical microscope further includes an observation system, an OCT system, and an optical-path connecting member. The observation system is used to observe, through the objective lens, the eye being illuminated by the illumination system. The OCT system is used to examine the eye E through the objective lens by means of OCT. The optical-path connecting member is located between the lens unit and the objective lens on the optical path of the illumination system to connect the optical path of the OCT system to the optical path of the illumination system.

With this configuration, in the ophthalmic surgical microscope used for the surgery of the eye, the aperture (diameter) of the optical element, through which the measurement light of the OCT system 20 passes, can be increased. Accordingly, it is possible to increase the scan range by the measurement light and the numerical aperture affecting the resolution of the measurement light of the OCT system. Besides, the optical path of the OCT system is connected to the optical path of the illumination system at a position between the lens unit and the objective lens. Such a design takes into account only the OCT system, thereby improving the accuracy of OCT examination. In addition, optical elements including at least the objective lens and the like are used in common. This results in a reduced number of optical elements, thus realizing a compact device. Further, similarly to the first embodiment, wide-range OCT examination can be performed with a high resolution.

According to the third embodiment, an ophthalmic surgical attachment is configured to be detachably attached to an ophthalmic surgical microscope which includes an objective lens, an illumination system, and an observation system. The illumination system includes a diaphragm irradiated with light from a light source, and a lens unit having one or more lenses configured to collimate the light having passed through the diaphragm into a parallel light flux, and is configured to irradiate the light having passed through the lens unit to the eye through the objective lens. The observation system is used to observe, through the objective lens, the eye being illuminated by the illumination system. The ophthalmic surgical attachment is used for examining the eye through the objective lens by OCT. The ophthalmic surgical attachment further includes a collimating lens, an optical scanner, an OCT lens, and an optical-path connecting member. The light from the OCT light source is split into measurement light and reference light. The interference optical system emits the measurement light, and causes the measurement light returning from the eye to interfere with the reference light to generate interference light. The collimating lens collimates the measurement light emitted from the interference optical system into a parallel light flux. The optical scanner two-dimensionally deflects the measurement light, which has been collimated into a parallel light flux by the collimating lens. The measurement light deflected by the optical scanner passes through the OCT lens. The optical-path connecting member is used to connect the optical path of the measurement light having passed through the OCT lens to the optical path of the illumination system. When the ophthalmic surgical attachment is attached to the ophthalmic surgical microscope, the optical-path connecting member is located at a position between the lens unit and the objective lens.

In this configuration, since the collimating lens, the optical scanner, the OCT lens, and the optical-path connecting member are modularized, they can be easily attached to and detached from the ophthalmic surgical microscope as an attachment. In addition, a relatively large physical space can be secured between the lens unit and the objective lens. Thus, it is possible to switch the ophthalmic surgical microscope to a device capable of OCT examination without influence on the optical system such as the positional deviation and the axial displacement of the lens unit, or the like.

### Fourth Embodiment

In the third embodiment, the illumination optical path and the OCT optical path are connected coaxially as in the first embodiment. Accordingly, vignetting may occur in the measurement light from the OCT system 20. In such a case, the range of OCT scan is limited, and the OCT system 20 cannot exert its full performance.

For this reason, in the fourth embodiment, the optical axis of the illumination system and the optical axis of the OCT system are arranged to be non-coaxial as in the second embodiment. The optical axis of the illumination system and the optical axis of the OCT system may be fixed to be non-coaxial, or they may be moved relatively to be adjusted as in the present embodiment.

An ophthalmic surgical microscope of the fourth embodiment has basically the same configuration as that of the third embodiment. In the following, the second embodiment is described with a focus on differences from the third embodiment.

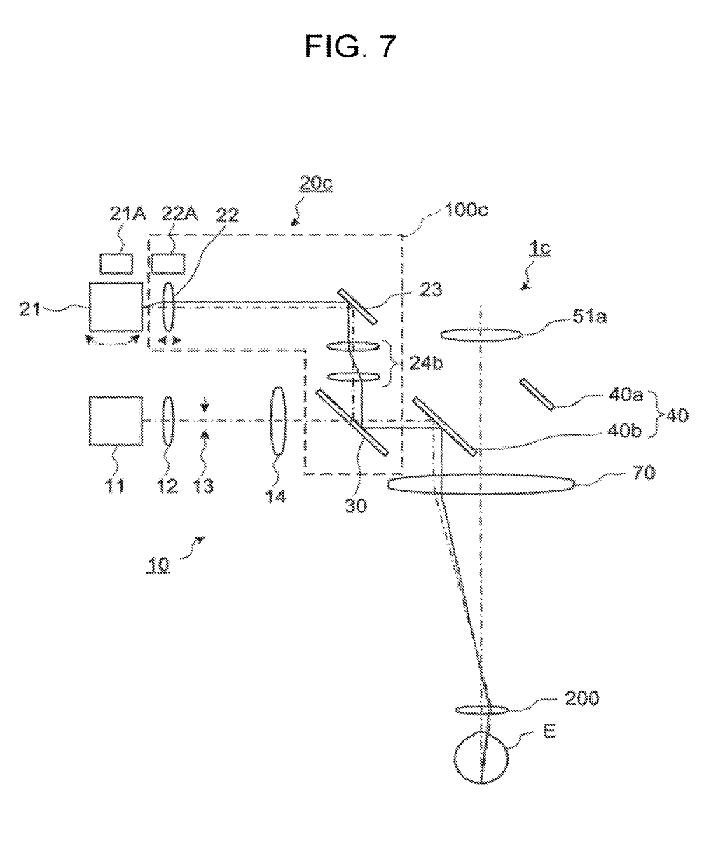

FIG. 7 illustrates the configuration of an optical system of an ophthalmic surgical microscope 1c of the fourth embodiment. In FIG. 7, like reference numerals designate like parts as in FIG. 6, and the redundant explanation may be omitted as appropriate.

The ophthalmic surgical microscope 1c of the fourth embodiment is different in configuration from the ophthalmic surgical microscope 1b of the third embodiment in the presence of the same optical-axis adjustment mechanism 21A as in the second embodiment. An OCT system 20c includes the interference optical system 21, the optical-axis adjustment mechanism 21A, the collimating lens 22, the focus adjustment mechanism 22A, the optical scanner 23, and the OCT lens 24b. An ophthalmic surgical attachment 100c includes the collimating lens 22, the focus adjustment mechanism 22A, the optical scanner 23, the OCT lens 24b, and the optical-path connecting member 30. The ophthalmic surgical attachment 100c may further include the interference optical system 21 and the optical-axis adjustment mechanism 21A. The ophthalmic surgical attachment 100c may further include an OCT light source.

The optical-axis adjustment mechanism 21A moves the optical axis of the illumination system 10 and that of the OCT system 20c relative to each other. For example, the optical-axis adjustment mechanism 21A changes the direction of measurement light emitted from the interference optical system 21 to relatively move the optical axis of the illumination system 10 and that of the OCT system 20c. The optical-axis adjustment mechanism 21A is basically the same as that of the second embodiment, and the same description is not repeated. The optical-axis adjustment mechanism 21A may change, for example, the direction of illumination light emitted from the illumination system 10 to move the optical axis of the illumination system 10 and that of the OCT system 20c relative to each other.

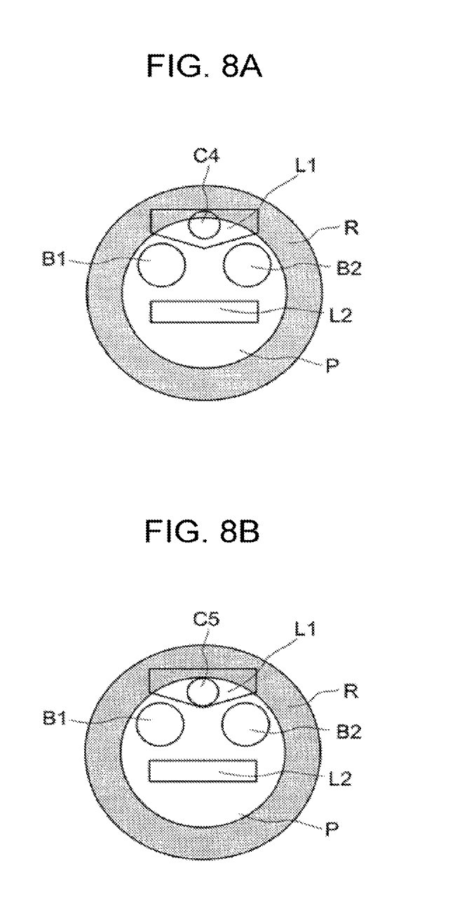

FIGS. 8A and 8B are diagrams for explaining the operation to observe the fundus of the eye E. FIG. 8A schematically illustrates the pupil of each optical system incident on the pupil of the eye E when vignetting occurs in measurement light due to positional deviation. FIG. 8B schematically illustrates the pupil of each optical system incident on the pupil of the eye E when the optical-axis adjustment mechanism 21A changes the direction of measurement light from the interference optical system 21. Like reference numerals designate like parts in FIGS. 8A and 8B, and the redundant explanation may be omitted as appropriate.

For example, when the position of the eye E has deviated with respect to the optical axis of the objective lens 70, the OCT pupil C4 of the OCT system 20c is arranged as illustrated in FIG. 8A, and vignetting may occur in the measurement light. Here, if the optical-axis adjustment mechanism 21A changes the direction of measurement light emitted from the interference optical system 21, as illustrated in FIG. 8B, OCT an pupil C5 of the OCT system 20c can be arranged to overlap the illumination pupil L1 in the pupil P. Thereby, it is possible to suppress the occurrence of vignetting of measurement light due to the positional deviation. Thus, the OCT system 20c can exert its full performance without limiting the range of OCT scan.

As described above, according to the fourth embodiment, in addition to the effects of the third embodiment, the effects by adjusting the optical axis can be achieved as in the second embodiment.

### Fifth Embodiment

In the fourth embodiment, an example is described in which the direction of measurement light emitted from the interference optical system is changed to shift the optical axis of the OCT system with respect to the optical axis of the illumination system; however, the configuration of the ophthalmic surgical microscope of the embodiment is not limited to this.

In the fifth embodiment, the optical scanner 23 and the OCT lens 24b are moved integrally in parallel to shift the optical axis of the OCT system with respect to the optical axis of the illumination system.

An ophthalmic surgical microscope of the fifth embodiment has basically the same configuration as that of the third embodiment. In the following, the fifth embodiment is described with a focus on differences from the third embodiment.

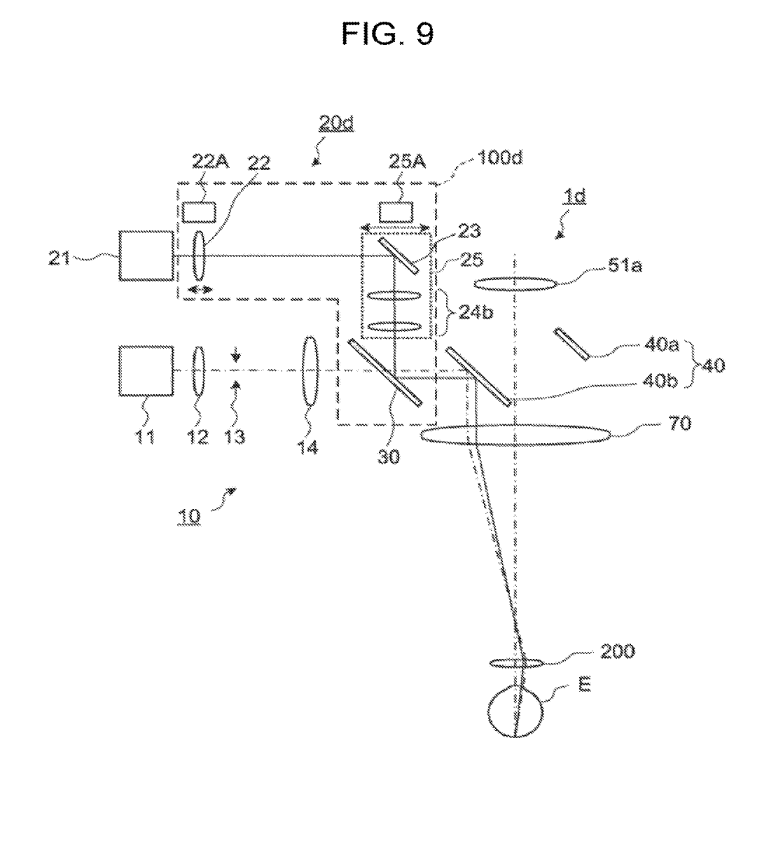

FIG. 9 illustrates the configuration of an optical system of an ophthalmic surgical microscope 1d of the fifth embodiment. In FIG. 9, like reference numerals designate like parts as in FIG. 6, and the redundant explanation may be omitted as appropriate.

The ophthalmic surgical microscope 1d of the fifth embodiment is different in configuration from the ophthalmic surgical microscope 1b of the third embodiment in that the optical scanner 23 and the OCT lens 24b are integrated as a scanner unit 25, and in the presence of an optical-axis adjustment mechanism 25A configured to move the scanner unit 25 in parallel. An OCT system 20d includes the interference optical system 21, the collimating lens 22, the focus adjustment mechanism 22A, the scanner unit 25 that includes the optical scanner 23 and the OCT lens 24b, and the optical-axis adjustment mechanism 25A. An ophthalmic surgical attachment 100d includes the collimating lens 22, the focus adjustment mechanism 22A, the scanner unit 25 that includes the optical scanner 23 and the OCT lens 24b, the optical-axis adjustment mechanism 25A, and the optical-path connecting member 30. The ophthalmic surgical attachment 100d may further include the interference optical system 21 and the optical-axis adjustment mechanism 21A. The ophthalmic surgical attachment 100d may further include an OCT light source.

The optical-axis adjustment mechanism 25A is configured to move the scanner unit 25 in parallel. For example, the optical-axis adjustment mechanism 25A moves the scanner unit 25 in parallel along a third direction that is parallel to the optical axis of the illumination system 10 and a fourth direction that is perpendicular to the third direction. Thus, the optical axis of the illumination system 10 and that of the OCT system 20d can be moved relatively to be arranged in different positions in the reflective surface of the deflector 40. In the fifth embodiment, the optical-path connecting member 30 is located on the parallel optical path where the illumination light is made into a parallel light flux. Therefore, the position of the scan plane in the eye E can be adjusted with a high precision, without being affected by the refraction of the lens unit 14. Moreover, as compared to the second embodiment or the fourth embodiment, the optical axis can be shifted in a wide range.

### Operations and Effects

According to the fifth embodiment, an ophthalmic surgical microscope includes an optical-axis adjustment mechanism (e.g., the optical-axis adjustment mechanism 25A) configured to move the optical axis of the illumination system (e.g., the illumination system 10) and the optical axis of the OCT system (e.g., the OCT system 20d) relative to each other. The optical-path connecting member is arranged in a position between the lens unit and the objective lens. The optical-axis adjustment mechanism changes the positions of the optical scanner and the OCT lens to move the optical axis of the illumination system and that of the OCT system.

In this configuration, the optical-path connecting member is located on the parallel optical path where the illumination light is made into a parallel light flux. Therefore, the position of the scan plane in the eye can be adjusted with a high precision, without being affected by the refraction of the lens unit. Further, it is possible to suppress the occurrence of vignetting of light in the OCT system. Thus, the OCT system can sufficiently exert the performance without limiting the range of OCT scan.

### Sixth Embodiment

The configuration, in which the optical axis of the OCT system can be shifted with respect to the optical axis of the illumination system, is not limited to that described in the fourth embodiment or the fifth embodiment.

In the sixth embodiment, there is provided a plane-parallel plate that can be arranged at a position between the optical scanner and the optical-path connecting member such that the incident surface is inclined with respect to the optical axis of the OCT system to, thereby, shift the optical axis of the OCT system with respect to the optical axis of the illumination system.

An ophthalmic surgical microscope of the sixth embodiment has basically the same configuration as that of the third embodiment. In the following, the sixth embodiment is described with a focus on differences from the third embodiment.

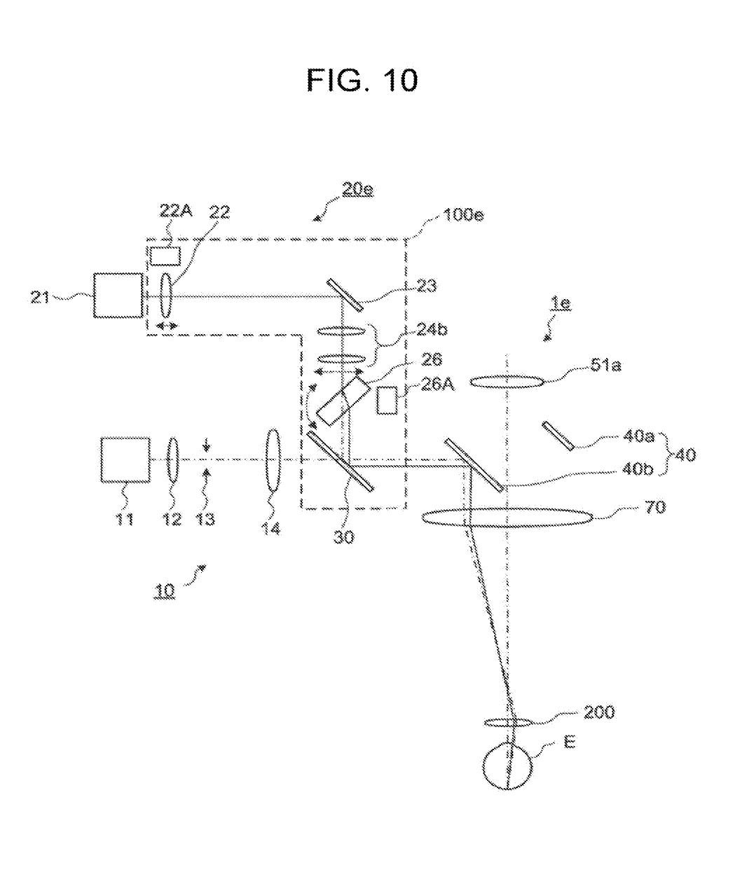

FIG. 10 illustrates the configuration of an optical system of an ophthalmic surgical microscope 1e of the sixth embodiment. In FIG. 10, like reference numerals designate like parts as in FIG. 6, and the redundant explanation may be omitted as appropriate.

The ophthalmic surgical microscope 1e of the sixth embodiment is different in configuration from the ophthalmic surgical microscope 1b of the third embodiment in the presence of a plane-parallel plate 26 and an optical-axis adjustment mechanism 26A. The plane-parallel plate 26 is removably inserted to a position between the optical scanner 23 and the optical-path connecting member 30. The optical-axis adjustment mechanism 26A is configured to move the plane-parallel plate 26. An OCT system 20e includes the interference optical system 21, the collimating lens 22, the focus adjustment mechanism 22A, the optical scanner 23, the OCT lens 24b, the plane-parallel plate 26, and the optical-axis adjustment mechanism 26A. An ophthalmic surgical attachment 100e includes the collimating lens 22, the focus adjustment mechanism 22A, the optical scanner 23, the OCT lens 24b, the plane-parallel plate 26, the optical-axis adjustment mechanism 26A, and the optical-path connecting member 30. The ophthalmic surgical attachment 100e may further include the interference optical system 21 and the optical-axis adjustment mechanism 21A. The ophthalmic surgical attachment 100e may further include an OCT light source.

The plane-parallel plate 26 is, for example, an optical element that is arranged such that the incident surface and the exit surface are parallel to each other, and transmits measurement light or return light thereof in the OCT system 20e. The plane-parallel plate 26 is configured to be removably inserted between the optical scanner 23 and the optical-path connecting member 30. When the plane-parallel plate 26 is inserted at a position between the optical scanner 23 and the optical-path connecting member 30, the incident surface is inclined with respect to the optical axis of the OCT system 20e. In FIG. 10, the plane-parallel plate 26 is configured to be removably inserted between the OCT lens 24b and the optical-path connecting member 30.

The optical-axis adjustment mechanism 26A is configured to move the plane-parallel plate 26 to insert/remove it to/from a position on the optical axis of the OCT system 20e between the OCT lens 24b and the optical-path connecting member 30. Thereby, the light having passed through the plane-parallel plate 26 is refracted, and the optical axis of the illumination system 10 and that of the OCT system 20e can be moved relatively to be arranged in different positions in the reflective surface of the deflector 40. In the sixth embodiment, the optical-path connecting member 30 is located on the parallel optical path where the illumination light is made into a parallel light flux. Therefore, the position of the scan plane in the eye E can be adjusted in stages without being affected by the refraction of the lens unit 14.

Incidentally, if the optical-axis adjustment mechanism 26A is configured to be capable of changing the direction of the incident surface of the plane-parallel plate 26 located between the OCT lens 24b and the optical-path connecting member 30, the position of the scan plane in the eye E can be adjusted more finely. In addition, the plane-parallel plate 26 may be fixed at a position between the OCT lens 24b and the optical-path connecting member 30 in advance. The optical-axis adjustment mechanism 26A may be capable of changing the direction of the incident surface of the plane-parallel plate 26.

### Operations and Effects

As described above, according to the sixth embodiment, an ophthalmic surgical microscope includes a plane-parallel plate (e.g., the plane-parallel plate 26) and an optical-axis adjustment mechanism (e.g., the optical-axis adjustment mechanism 26A). The plane-parallel plate is configured to be removably inserted to a position between the optical scanner and the optical-path connecting member and is arranged such that the incident surface is inclined with respect to the optical axis of the OCT system (e.g., the OCT system 20e). The optical-axis adjustment mechanism is configured to insert/remove the plane-parallel plate to/from the position between the optical scanner and the optical-path connecting member. The optical-path connecting member is located between the lens unit and the objective lens. The optical-axis adjustment mechanism inserts/removes the plane-parallel plate to/from between the optical scanner and the optical-path connecting member to move the optical axis of the illumination system and the optical axis of the OCT system relative to each other.

Besides, the ophthalmic surgical microscope of the embodiment may include a plane-parallel plate (e.g., the plane-parallel plate 26) and an optical-axis adjustment mechanism (e.g., the optical-axis adjustment mechanism 26A). The plane-parallel plate is arranged between the optical scanner and the optical-path connecting member, and has an incident surface the direction of which can be changed with respect to the optical axis of the OCT system. The optical-axis adjustment mechanism is configured to change the direction of the incident surface. The optical-path connecting member is located between the lens unit and the objective lens. The optical-axis adjustment mechanism changes the direction of the incident surface to move the optical axis of the illumination system and that of the OCT system relative to each other.

In any of the configurations described above, the optical-path connecting member is located on the parallel optical path where the illumination light is made into a parallel light flux. Therefore, the position of the scan plane in the eye can be adjusted with a high precision, without being affected by the refraction of the lens unit. Further, it is possible to suppress the occurrence of vignetting of light in the OCT system. Thus, the OCT system can sufficiently exert the performance without limiting the range of OCT scan.

### Seventh Embodiment

If provided with a beam splitter arranged on the observation optical axis in at least part of the deflector 40, the ophthalmic surgical microscope having the optical-axis adjustment mechanism as described above becomes capable of switching between the coaxial state where the optical axis of the OCT system and the observation optical axis are coaxial and the non-coaxial state where they are non-coaxial.

An ophthalmic surgical microscope of the seventh embodiment has basically the same configuration as that of the sixth embodiment. In the following, the seventh embodiment is described with a focus on differences from the sixth embodiment.

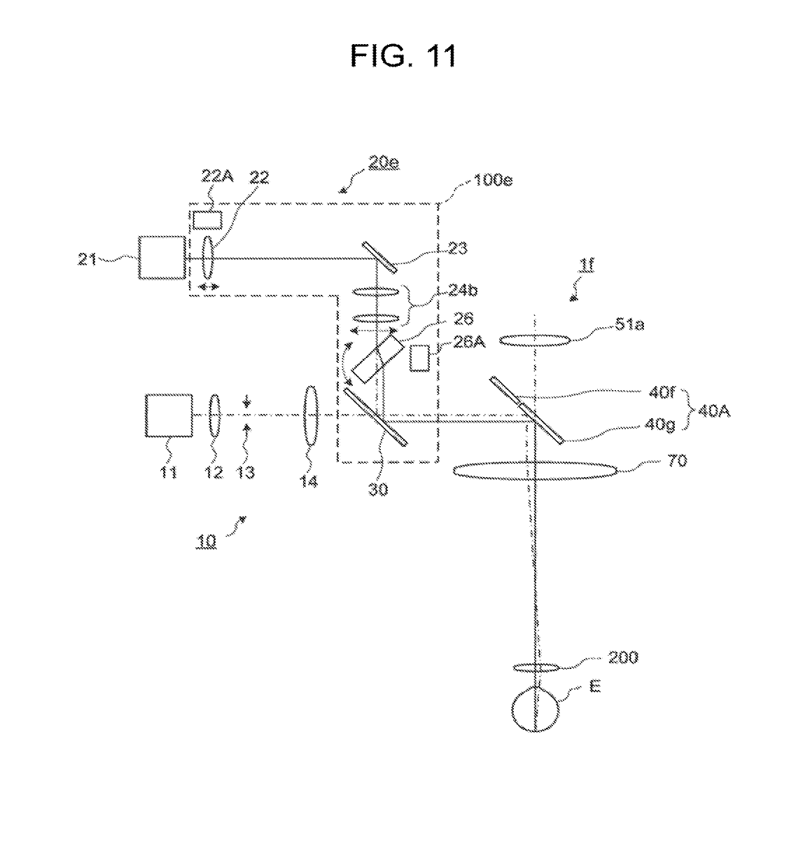

FIG. 11 illustrates the configuration of an optical system of an ophthalmic surgical microscope if of the seventh embodiment. In FIG. 11, like reference numerals designate like parts as in FIG. 10, and the redundant explanation may be omitted as appropriate.

The ophthalmic surgical microscope if of the seventh embodiment is different in configuration from the ophthalmic surgical microscope 1e of the sixth embodiment in the presence of a deflector 40A provided in place of the deflector 40. The deflector 40A includes a reflecting member 40f and a beam splitter 40g. The beam splitter 40g is arranged on the observation optical axis of the observation system 50 (the left observation optical axis 50La or the right observation optical axis 50Ra), and connects the optical path of the illumination system 10 and that of the observation system 50 to each other. Note that the deflector 40A may connect the optical path of the illumination system 10 and that of the observation system 50 by using another optical element such as a dichroic mirror, a half mirror, or the like instead of the beam splitter 40g.

In this configuration, the optical-axis adjustment mechanism 26A adjusts the optical axis of the OCT system 20e with respect to the optical axis of the illumination system 10. Thereby, the optical axis of the OCT system 20e can be adjusted to be coaxial or non-coaxial with the observation optical axis.

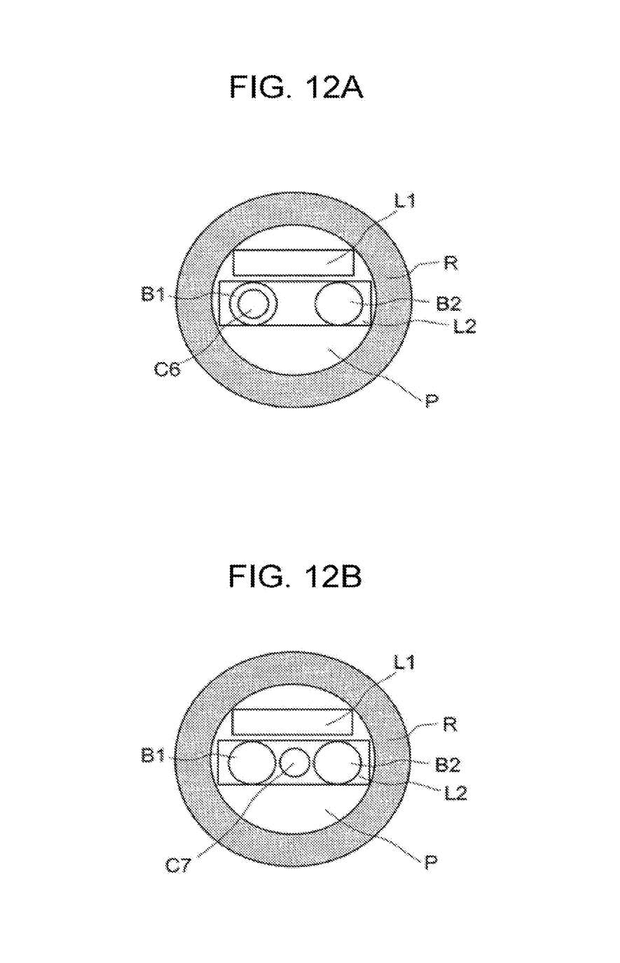

FIGS. 12A and 12B are diagrams for explaining the operation to observe the fundus of the eye E. FIG. 12A schematically illustrates the pupil of each optical system incident on the pupil of the eye E when the optical axis of the OCT system 20e is adjusted to be coaxial with the observation optical axis. FIG. 12B schematically illustrates the pupil of each optical system incident on the pupil of the eye E when the optical axis of the OCT system 20e is adjusted to be non-coaxial with the observation optical axis. Like reference numerals designate like parts in FIGS. 12A and 12B, and the redundant explanation may be omitted as appropriate.

When the optical axis of the OCT system 20e is adjusted to be coaxial with the observation optical axis, as illustrated in FIG. 12A, an OCT pupil C6 of the OCT system 20e is arranged to overlap the observation pupil B1 of the left observation system 50L. At this time, it becomes possible to perform an examination by OCT under conditions similar to those in the observation by the observation system 50.

On the other hand, when the optical axis of the OCT system 20e is adjusted to be non-coaxial with the observation optical axis, as illustrated in FIG. 12B, an OCT pupil C7 of the OCT system 20e is arranged between the observation pupil B1 of the left observation system 50L and the observation pupil B2 of the right observation system 50R. In this case, the occurrence of vignetting can be suppressed as described above. Thus, the OCT system 20e can sufficiently exert the performance without limiting the range of OCT scan.

### MODIFICATIONS

The embodiments described above are mere examples for embodying or carrying out the present invention, and therefore susceptible to several modifications and variations (omission, substitution, addition, etc.), all coming within the scope of the invention.

The various features of the above embodiments may be combined in arbitrary ways. For example, in a system which can use two or more of the first to seventh embodiments, desired one of the two or more embodiments can be selectively used by switching the operation mode.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ophthalmic surgical microscope, comprising:
an objective lens;
an illumination system including a diaphragm configured to be irradiated with light from an illumination light source, and a lens unit including one or more lenses configured to make the light having passed through the diaphragm into a parallel light flux, wherein the illumination system is configured to irradiate an eye with the light having passed through the lens unit through the objective lens;
an observation system configured for observing the eye being irradiated by the illumination system through the objective lens;
an optical coherence tomography (OCT) system configured for examining the eye by OCT through the objective lens; and
an optical-path connecting member located between the diaphragm and the lens unit or between the lens unit and the objective lens to connect an optical path of the OCT system to an optical path of the illumination system.

2. The ophthalmic surgical microscope of claim 1, further comprising a deflector, which is located between the optical-path connecting member and the objective lens, and is configured to deflect light in the optical path of the illumination system and light in the optical path of the OCT system toward the objective lens.

3. The ophthalmic surgical microscope of claim 2, wherein the deflector includes a beam splitter, which is located on an optical path of the observation system.

4. The ophthalmic surgical microscope of claim 3, wherein the beam splitter is configured to coaxially connect the optical path of the observation system and the optical path of the OCT system with each other.

5. The ophthalmic surgical microscope of claim 1, wherein an optical axis of the illumination system and an optical axis of the OCT system are arranged to be non-coaxial.

6. The ophthalmic surgical microscope of claim 1, wherein
the OCT system split comprises:
an interference optical system configured to split light from an OCT light source into measurement light and reference light, emit the measurement light, and interfere the measurement light returning from the eye with the reference light to generate interference light;
a collimating lens configured to collimate the measurement light emitted from the interference optical system into a parallel light flux;
an optical scanner configured to two-dimensionally deflect the measurement light, which has been collimated into a parallel light flux by the collimating lens; and
an OCT lens located between the optical scanner and the optical-path connecting member.

7. The ophthalmic surgical microscope of claim 6, further comprising a focus adjustment mechanism configured to move the collimating lens along an optical axis of the OCT system.

8. The ophthalmic surgical microscope of claim 6, further comprising an ophthalmic surgical attachment configured to be detachably attached to the ophthalmic surgical microscope,
wherein the collimating lens, the optical scanner, the OCT lens, and the optical-path connecting member are located in the ophthalmic surgical attachment.

9. The ophthalmic surgical microscope of claim 1, further comprising an optical-axis adjustment mechanism configured to move an optical axis of the illumination system and an optical axis of the OCT system relative to each other.

10. The ophthalmic surgical microscope of claim 9, wherein
the OCT system split comprises an interference optical system configured to split light from an OCT light source into measurement light and reference light, emit the measurement light, and interfere the measurement light returning from the eye with the reference light to generate interference light, and
the optical-axis adjustment mechanism is configured to change direction of the measurement light emitted from the interference optical system to move the optical axis of the illumination system and the optical axis of the OCT system relative to each other.

11. The ophthalmic surgical microscope of claim 6, further comprising an optical-axis adjustment mechanism configured to move an optical axis of the illumination system and an optical axis of the OCT system relative to each other,
wherein the optical-path connecting member is located between the lens unit and the objective lens, and
the optical-axis adjustment mechanism is configured to change positions of the optical scanner and the OCT lens to move the optical axis of the illumination system and the optical axis of the OCT system relative to each other.

12. The ophthalmic surgical microscope of claim 6, further comprising:
a plane-parallel plate configured to be removably inserted between the optical scanner and the optical-path connecting member, and arranged such that an incident surface thereof is inclined with respect to the optical axis of the OCT system when the plane-parallel plate is inserted between the optical scanner and the optical-path connecting member, and
an optical-axis adjustment mechanism configured to insert or remove the plane-parallel plate to or from a position between the optical scanner and the optical-path connecting member,
wherein the optical-path connecting member is located between the lens unit and the objective lens, and
the optical-axis adjustment mechanism is configured to insert or remove the plane-parallel plate to or from the position between the optical scanner and the optical-path connecting member to move an optical axis of the illumination system and an optical axis of the OCT system relative to each other.

13. The ophthalmic surgical microscope of claim 6, further comprising:
a plane-parallel plate located between the optical scanner and the optical-path connecting member, and configured such that direction of an incident surface thereof is variable with respect to an optical axis of the OCT system, and
an optical-axis adjustment mechanism configured to change the direction of the incident surface,
wherein the optical-path connecting member is located between the lens unit and the objective lens, and
the optical-axis adjustment mechanism is configured to change the direction of the incident surface to move an optical axis of the illumination system and the optical axis of the OCT system relative to each other.

14. An ophthalmic surgical attachment configured to be detachably attached to an ophthalmic surgical microscope including:
an objective lens;
an illumination system including a diaphragm configured to be irradiated with light from an illumination light source, and a lens unit including one or more lenses configured to make the light having passed through the diaphragm into a parallel light flux, wherein the illumination system is configured to irradiate the light having passed through the lens unit to an eye through the objective lens; and
an observation system configured for observing the eye being irradiated by the illumination system through the objective lens,
the ophthalmic surgical attachment for examining the eye by optical coherence tomography (OCT) through the objective lens comprising:
a collimating lens configured to collimate measurement light emitted from an interference optical system into a parallel light flux, wherein the interference optical system is configured to emit the measurement light, and cause the measurement light returning from the eye to interfere with reference light to generate interference light, and the measurement light and the reference light are obtained by splitting light from an OCT light source;
an optical scanner configured to two-dimensionally deflect the measurement light, which has been collimated into a parallel light flux by the collimating lens;
an OCT lens through which the measurement light, which has been deflected by the optical scanner, passes; and
an optical-path connecting member configured for connecting an optical path of the measurement light, which has passed through the OCT lens, to an optical path of the illumination system,
wherein when the ophthalmic surgical attachment is attached to the ophthalmic surgical microscope, the optical-path connecting member is located between the diaphragm and the lens unit or between the lens unit and the objective lens.
